(19) United States
(12) Patent Application Publication
(10) Pub. No.: US 2004/0122045 A1
(43) Pub. Date: Jun. 24, 2004

(54) Timing curation of administration of adenosine A1/A2 agonist for cardioprotection

(75) Inventors: Zhelong Xu, Chapel Hill, NC (US); James M. Downey, Mobile, AL (US)

Correspondence Address: ROSS J. OEHLER, AVENTIS PHARMACEUTICALS INC., ROUTE 202-206, MAIL CODE: D303A, BRIDGEWATER, NJ, 08807, US

(73) Assignee: Aventis Pharmaceuticals Inc., Bridgewater, NJ

(21) Appl. No.: 10/696,527

(22) Filed: Oct. 29, 2003

Related U.S. Application Data
(63) Continuation of application No. PCT/US02/14228, filed on May 3, 2002.
(60) Provisional application No. 60/288,936, filed on May 4, 2001.

(30) Foreign Application Priority Data
Aug. 17, 2001 (GB) 0120124.3

(51) Int. Cl. A61K 31/4745

(52) U.S. Cl. 514/303

(57) ABSTRACT

This invention is directed to methods of providing cardioprotection in a patient in need thereof comprising administering to said patient a pharmaceutically effective amount of a compound having adenosine A1/A2 agonistic activity, or a pharmaceutically acceptable salt thereof, or a pharmaceutical composition thereof, beginning at a time less than 10 minutes after the onset reperfusion, at reperfusion, and ten minutes or more before reperfusion, and continuing for a period of more than 30 minutes following the onset of reperfusion.

This application is a continuation of International Application No. PCT/US02/14228, filed May 3, 2002, which claims the benefit of Provisional Application No. 60/288,936, file May 4, 2001.

### FIELD OF THE INVENTION

This invention is directed to methods of providing cardioprotection in a patient in need thereof comprising administering a pharmaceutically effective amount of a compound having adenosine A1/A2 agonistic activity.

### SUMMARY OF THE INVENTION

This invention is directed to methods of providing cardioprotection in a patient in need thereof comprising administering to said patient a pharmaceutically effective amount of a compound having adenosine A1/A2 agonistic activity, beginning at a time less than 10 minutes after the onset reperfusion, at reperfusion, and ten minutes or more before reperfusion, and continuing for a period of more than 30 minutes following the onset of reperfusion.

### DETAILED DESCRIPTION OF THE INVENTION

As used above, and throughout the description of the invention, the following terms, unless otherwise indicated, shall be understood to have the following meanings.

“Patient” includes both human and other mammals.

“Effective amount” is meant to describe an amount of compound having A1/A2 agonistic activity used in the method according to the present invention which is effective in producing the desired therapeutic effect.

“Cardioprotection” means protecting against or reducing damage to the myocardium, for example prior to, during or after an ischemic attack, during reperfusion or prior to, during or after cardiac surgery.

“Adenosine A1/A2 agonist” or “compound having adenosine A1/A2 agonistic activity” means a compound that is an agonist for both the A1 and A2 subtypes of adenosine receptors, for example, AMP 579.

“AMP 579” is [1S-[1α,2β,3β,4α(S*)]]-4-[7-[[3-chloro-2-thienyl)methyl]propyl]amino]-3H-imidazo[4,5-b]pyridin-3-yl]-N-ethyl-2,3-dihydroxycyclopentanecarboxamide, or

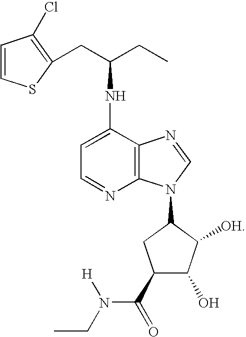

The effects of timing and duration of treatment with the adenosine A₁/A₂ receptor agonist AMP 579 on ischemia/reperfusion injury were investigated in in situ rabbit hearts subjected to 30 min regional ischemia/3 h reperfusion. AMP 579 was infused either 10 min before reperfusion and continuing for 40 min, at the onset of reperfusion for 70 min, or 10 min after reperfusion for 70 min. In untreated hearts 36.4±3.1% of the risk zone infatrcted. Protection was observed in only the group with 70 min of AM 579 started at reperfusion (13.0±1.9%, p<0.05). Adenosine, 150, 300, or 400 μg/kg/min, was infused for 70 min starting 10 min before reperfusion. No protection was seen. Therefore, AMP 579 must be present at the moment of reperfusion and for more than 30 min thereafter to protect. We were unable to duplicate AMP 579's anti-infarct effect with adenosine.

The novel adenosine A₁/A₂ receptor agonist AMP 579 {cyclopentanecarboxamide, 4-[4-[[(1R)-1-[(3-chloro-2-thienyl)methyl]propyl]amino]-7H-pyrrolo[2,3,-d]pyrimidin-7-yl]-N-ethyl-2,3-dihydroxy-, (1S,2R,3S,4R)-(9Cl)}, with Kᵢ=5 and 56 nM for A₁ and A₂ₐ receptor subtypes, respectively (Smits, G. J., McVey, M., Cox, B. F., Perrone, M. H., Clark, K. L., 1998, Cardioprotective effects of the novel adenosine A₁/A₂ receptor agonist AMP 579 in a porcine model of myocardial infarction. J Pharmacol Exp Ther 286, 611-618) (hereinafter, “Smits, et al.”), has been demonstrated to protect the heart from infarction following prolonged ischemia and reperfusion in a variety of animal species (Smits, et al.; McVey, M. J., Smits, G. J., Cox, B. F., Kitzen, J. M., Clark, K. L., Perrone, M. H., 1999. Cardiovascular pharmacology of the adenosine A₁/A₂-receptor agonist AMP 579: coronary hemodynamic and cardioprotective effects in the canine myocardium. J Cardiovasc Pharmacol 33, 703-710 (hereinafter, “McVey, et al.”); Budde, J. M., Velez, D. A., Zhao, Z. -Q., Clark, K. L., Morris, C. D., Muraki, S., Guyton, R. A., Vinten-Johansen, J., 2000. Comparative study of AMP579 and adenosine in inhibition of neutrophil-mediated vascular and myocardial injury during 24 h of reperfusion. Cardiovasc Res 47, 294-305 (hereinafter, “Budde et al.”). An intriguing and potentially clinically significant effect of the agent was limitation of infarction even when administration of AMP 579 was started just before onset of reperfusion (Budde, et al.). These results indicate that AMP 579 could be a therapeutic agent in the setting of acute myocardial infarction. In the published studies AMP 579 was infused for 70 min starting 10 min prior to reperfusion. In a prior study from this laboratory the dose required for protection was investigated (Xu, Z., Yang, X. -M., Cohen, M. V., Neumann, T., Heusch, G., Downey, J. M., 2000. Limitation of infarct size in rabbit hearts by the novel adenosine receptor agonist AMP 579 administered at reperfusion. J Mol Cell Cardiol 32, 2339-2347) (hereinafter, “Xu, et al.”). We found that 3 μg/kg/min was protective, but that 1 jig/kg/min was not. In the present study we have explored the timing requirements for AMP 579's protection. Because of the hypotensive side effect of AMP 579, it would be desirable to give it for as short a period as possible. In addition we wanted to know if it would still be effective if started shortly after reperfusion had occurred. Several investigators have noted that the protection involves adenosine receptor activation (Smits et al.; McVey et al.; Xu et al.), but curiously a recent study failed to duplicate AMP 579's protection with an infusion of adenosine (Budde et al.). Because adenosine is readily available for clinical use and because it has been reported to limit infarct size in previous studies (Norton, E. D., Jackson, E. K., Virmani, R., Forman, M. B., 1991. Effect of intravenous adenosine on myocardial reperfusion injury in a model with low myocardial collateral blood flow. Am Heart J 122, 1283-1291 (hereinafter, “Norton, et al.”); Olafsson, B., Forman, M. B., Puett, D. W., Pou, A., Cates, C. U., Friesinger, G. C., Virmani, R., 1987. Reduction of reperfusion injury in the canine preparation by intracoronary adenosine: importance of the endothelium and the no-reflow phenomenon. Circulation. 76, 1135-1145 (hereinafter, “Olafsson, et al.”), we tested whether adenosine could duplicate AMP579's anti-infarct effect in the present model.

Materials and Methods

This study was performed in accordance with The Guide for the Care and Use of Laboratory Animals (National Academy Press, Washington, D.C., 1996).

Surgical Preparation

New Zealand White rabbits of either sex weighing 1.6-2.5 kg were anesthetized with pentobarbital (30 mg/kg iv), intubated through a tracheotomy, and ventilated with 100% oxygen via a positive pressure respirator (MD industries, Mobile, Ala.). The ventilation rate and tidal volume were adjusted to maintain pCO₂ and pH in the physiological range. Body temperature was maintained at 38-39° C. A catheter was inserted into the left carotid artery for monitoring blood pressure. Another catheter was inserted into the right jugular vein for drug infusion. A left thoracotomy was performed in the fourth intercostal space, and the pericardium was opened to expose the heart. A 2-0 silk suture on a curved taper needle was passed through the myocardium around a prominent branch of the left coronary artery. The ends of the suture were passed through a small piece of soft vinyl tubing to form a snare. Ischemia was induced by pulling the snare and then fixing it by clamping the tube with a small hemostat. Ischemia was confirmed by appearance of cyanosis. Reperfusion was achieved by releasing the snare and was confirmed by visible hyperemia on the ventricular surface.

After 3 h of reperfusion, the rabbit was given an overdose of pentobarbital and the heart was quickly removed from the chest, mounted on a Langendorff apparatus, and perfused with saline to wash out blood. Then the coronary artery was reoccluded, and 5 ml of 0.1% zinc/cadmium sulfide particles (1-10 μm diameter, Duke Scientific Corp, Palo Alto, Calif.) were infused into the perfusate to demarcate the risk zone as the area of tissue without fluorescence. The heart was weighed, frozen, and cut into 2.5-mm-thick slices. The slices were incubated in 1% triphenyltetrazolium chloride (TTC) in sodium phosphate buffer at 37° C. for 20 min. The slices were immersed in 10% formalin to enhance the contrast between stained (viable) and unstained (necrotic) tissue and then squeezed between glass plates spaced exactly 2 mm apart. The myocardium at risk was identified by illuminating the slices with ultraviolet light. The infarcted and risk zone areas were traced on a clear acetate sheet and quantified with planimetry by an investigator blinded to the treatment. The areas were converted into volumes by multiplying the areas by slice thickness. Infarct size is expressed as a percentage of the risk zone.

Experimental Protocols

Eight groups of rabbits were subjected to 30 min of regional ischemia followed by 3 h of reperfusion. The control group received no drug treatment. All groups receiving AMP 579 received a bolus injection of 30 μg/kg iv followed by an infusion of 3 μg/kg/min. In the first 2 AMP 579 groups infusion of AMP 579 was started 10 min before reperfusion and continued for 30 (AMP 30 min) or 40 (AMP 40 min) min. In the next 2 groups AMP 579 was infused for 70 min but the infusion was started either with reperfusion (AMP 70 min, onset) or 10 min after reperfusion (AMP 70 min, late). The next three groups received an infusion of intravenous adenosine for 70 min starting 10 min prior to reperfusion, the original schedule that was protective for AMP 579 in our previous study (Xu et al.). Because the plasma half-life of adenosine is much shorter than that for AMP 579, a bolus dose was not needed. Adenosine's hemodynamic effects plateaued several minutes after the onset of infusion well before reperfusion. These rabbits received 150 (Ado 150), 300 (Ado 300), or 400 (Ado 400) μg/kg/min of adenosine intravenously.

Chemicals

AMP 579 was obtained from Aventis Pharma and dissolved in small volumes of dimethylsulfoxide (DMSO) which had no independent effect on infarction. The DMSO solution was then diluted in saline and the final concentration of DMSO was <0.1%. Adenosine was obtained from Sigma and dissolved in normal saline.

Statistics

All data are expressed as means±S.E.M. One-way ANOVA combined with Scheffé's post hoc test was used to test for differences in baseline hemodynamics and infarct size among groups. ANOVA with replication was used to test for changes in hemodynamics during an experiment within each group. A p value of less than 0.05 was considered to be significant.

Results

Baseline heart rate and mean arterial pressure were not different among the eight groups (Tables 1 and 2). Table 1 shows hemodynamic data for AMP 579. (Mean±S.E.M). Hemodynamics were measured at the end of 30 min of ischemia. Abbreviations used in Table 1: AMP (30, 40 min)=administration of AMP 579 starting 10 min before reperfusion for a total of 30, 40 min, respectively; AMP (70 min, late)=administration of AMP 579 starting 10 min after reperfusion and lasting for 70 min; AMP (70 min, onset)=administration of AMP 579 starting at onset of reperfusion and extending for 70 min; HR=heart rate; MAP=mean arterial pressure; Rep=reperfusion. Table 2 shows hemodynamic data for adenosine. Hemodynamics were measured at the end of 30 minutes of ischemia. Abbreviations are the same as for Table 1. Additionally, Adenosine 150, 300, 400=adenosine infusion of 150, 300, 400 μg/kg/min, respectively. Ischemia lowered arterial pressure in all groups. AMP 579 produced an additional mild hypotensive effect when the infusion was commenced before the onset of reperfusion (AMP 30 min and AMP 40 min groups). The lowest dose of adenosine (150 μg/kg/min) produced comparable mild hypotension, whereas the 2 higher doses led to significantly greater degrees of hypotension. The latter precluded further increases in dose.

There were no significant differences in body weight, heart weight and risk zone size among the groups (Tables 3 and 4). Table 3 shows infarct size data for AMP 579 (Mean±S.E.M., p<0.05 vs. control). Abbreviations in Table 3 are the same as for Table 1. Additionally, n=number of rabbits in each group. Table 4 shows infarct size data for adenosine (Mean±S.E.M.). Abbreviations in Table 4 are the same as Tables 2 and 3. Infarct size in the control hearts was 36.4±3.1%. When administered before reperfusion, neither a 30 nor 40 min infusion of AMP 579 reduced infarct size (30.1±1.4 and 24.8±5.9%, respectively), indicating the importance of the duration of treatment. In the 70 min groups, AMP 579 was not protective when infused shortly after the onset of reperfusion (27.6±7.2%), but was very effective when present at the onset of reperfusion (13.0±1.9%, p<0.05 vs. control). While the data indicate that AMP 579 protects against some critical event that occurs in the first few minutes of reperfusion, they also indicate that a continued presence of the drug is needed for more than 30 min after reperfusion has occurred.

We next tested to see if adenosine infusion could duplicate AMP 579's protection when given for 70 min beginning 10 min before the onset of reperfusion. Three doses of adenosine were tested, but as can be seen in Table 5, none of the doses was protective. Table 5 shows data at 100 minutes of exposure starting 10 minutes before reperfusion. Significant hypotension prevented further increases in doses beyond 400 μg/kg/min.

Discussion

In the present study, AMP 579 had salutary effects only when administered at the onset of reperfusion and continued for a relatively long time (70 min). Neither of the shorter treatments (30 and 40 min) was protective even though the infusions started 10 min prior to reperfusion. These results were somewhat surprising. Delaying the onset of administration of AMP 579 to just 10 min after reperfusion completely abolished protection. In our original study we protected the rabbit heart with a 70-min infusion starting 10 prior to reperfusion (Xu et al.). It is doubtful that significant drug would have entered the ischemic zone prior to reperfusion in that study because collateral flow is negligible in the rabbit. Nevertheless we could not eliminate the possibility that AMP 579 might have exerted its protection on events occuring in the last 10 min of ischemia. In the present study the coronary snare was released immediately following the loading bolus of AMP 579 in the AMP 70 min, onset group, and protection was almost the same as that seen in the previous study (12.3±1.0% previously (Xu et al.) vs. 13.0±1.9% now). That would imply that AMP 579 was protecting against some toxic event in the first few minutes of reperfusion, but it also had to be present for more than 30 additional minutes. Clearly the timing and duration of administration are crucial for the protective effect of AMP 579.

AMP 579 has been demonstrated to protect the heart against myocardial infarction following ischemia/reperfusion when administered before reperfusion (Smits et al.; Budde et al.). The present study revealed that AMP 579 must be present at the moment of reperfusion to be protective. Delaying its administration to 10 min after reperfusion completely abolished the protection suggesting that AMP 579 acts to eliminate a reperfusion type of injury. It has been proposed for some time that the act of reperfusion itself induces some form of injury (Braunwald, E., Kloner, R. A., 1985. Myocardial reperfusion: a double-edged sword? J Clin Invest 76, 1713-1719). Although free radicals or calcium flooding have variously been proposed as mediators of this reperfusion injury (Piper, H. M., García-Dorado, D., Ovize, M., 1998. A fresh look at reperfusion injury. Cardiovasc Res 38, 291-300), direct proof that either is contributing to infarction has been difficult to demonstrate. We have recently found that AMP 579 greatly attenuates the onset of contracture that accompanies reperfusion (Xu, Z., Downey, J. M., Cohen, M. V., 2001, AMP 579 reduces contracture and limits infarction in rabbit heart by activating adenosine A₂ receptors. J Cardiovasc Pharmacol 38, 474-481) (hereinafter, “Xu, et al. II”) which would be compatible with the hypothesis that it reduced calcium entry upon reperfusion. At the same time we also found in a chick cardiomyocyte model that AMP 579 can suppress the burst of free radicals seen at reperfusion (Xu, Z., Cohen, M. V., Downey, J. M., Vanden Hoek, T. L., Yao, Z., 2001a. Attenuation of oxidant stress during reoxygenation by AMP 579 in cardiomyocytes. Am J Physiol 281, H2585-H2589) (hereinafter, “Xu, et al. III”). These results strongly suggest that AMP 579 not only protects the heart from ischemic injury but also from a reperfusion injury per se. But if the protection is against an event that occurs in the first 10 minutes of reperfusion, then it is not clear why the drug has to be present for more than 30 additional minutes after reperfusion.

Several reports indicate that AMP 579's protection can be blocked by an adenosine receptor blocker (Smits et al.; McVey et al.; Xu et al.). Furthermore the protective effect seems to involve the A₂ receptor subtype (Smits et al.; Xu et al. II). Yet in one recent study adenosine failed to duplicate AMP 579's protection (Budde et al.). There have been several reports that adenosine started just prior to reperfusion could limit infarct size (Norton et al.; Olafsson et al.). However, those results have been controversial since others have failed to reproduce them (Goto, M., Miura, T., Iliodoromitis, E. K., O'Leary, E. L., Ishimoto, R., Yellon, D. M., Iimura, O., 1991. Adenosine infusion during early reperfusion failed to limit myocardial infarct size in a collateral deficient species. Cardiovasc Res 25, 943-949; Vander Heide, R. S., Reimer, K. A., 1996. Effect of adenosine therapy at reperfusion on myocardial infarct size in dogs. Cardiovasc Res 31, 711-718). In the present study we gave adenosine at 3 different doses, but none was protective even when infused with the same timing as that used when AMP 579 was protective. Both AMP 579 and adenosine lower blood pressure primarily through activation of A₂ adenosine receptors on blood vessels causing the latter to dilate. Because the degree of hypotension was similar in the low dose adenosine and the AMP 579 groups, we would assume that the A₂ receptor occupation was similar in both cases. Yet adenosine did not protect. In our recent study with chick cardiomyocytes 100 μM adenosine (a receptor saturating concentration) failed to block the burst of free radicals at reperfusion, while 1 μM AMP 579 virtually eliminated it (Xu et al. III). Thus while adenosine receptor activation is required for AMP 579's protection, adenosine itself will not duplicate the effect. This would indicate that something else in the molecule acts synergistically with adenosine receptor stimulation to produce the protection.

In summary, we have demonstrated that AMP 579 must not only be present in the first minutes of reperfusion but must remain present for more than 30 minutes following the onset of reperfusion to achieve an anti-infarct effect in the open-chest rabbit model. Curiously adenosine administered with the same schedule as was protective for AMP 579 produced no salvage of myocardial tissue.

TABLE 1

| | | | | |
|---|---|---|---|---|
| Baseline | Ischemia | Rep 30′ | Rep 90′ | Rep 180′ |

HR (beats/min)

| | | | | | |
|---|---|---|---|---|---|
| Control | 275 ± 2 | 265 ± 9 | 266 ± 11 | 272 ± 12 | 266 ± 10 |
| AMP (30 min) | 271 ± 3 | 247 ± 5 | 241 ± 7 | 256 ± 11 | 265 ± 6 |
| AMP (40 min) | 271 ± 11 | 268 ± 7 | 250 ± 14 | 268 ± 13 | 268 ± 10 |
| AMP (70 min, late) | 285 ± 3 | 281 ± 1 | 243 ± 8 | 240 ± 6 | 259 ± 10 |
| AMP (70 min, onset) | 262 ± 6 | 270 ± 9 | 233 ± 9 | 235 ± 16 | 264 ± 10 |
| MAP (mmHg) | | | | | |
| Control | 90.3 ± 3.7 | 78.3 ± 4.3 | 72.0 ± 6.5 | 78.0 ± 6.0 | 62.4 ± 4.4 |
| AMP (30 min) | 91.8 ± 2.8 | 73.7 ± 3.9 | 74.8 ± 5.1 | 77.7 ± 5.7 | 77.9 ± 5.2 |
| AMP (40 min) | 99.3 ± 2.4 | 76.8 ± 6.6 | 70.4 ± 8.1 | 83.5 ± 5.0 | 81.8 ± 4.5 |
| AMP (70 min, late) | 96.9 ± 2.9 | 84.6 ± 4.5 | 57.4 ± 5.6 | 69.0 ± 4.9 | 68.1 ± 3.6 |
| AMP (70 min, onset) | 98.6 ± 3.2 | 89.0 ± 3.2 | 55.6 ± 3.9 | 70.8 ± 4.5 | 79.6 ± 3.9 |

TABLE 2

| | | | | |
|---|---|---|---|---|
| Baseline | Ischemia | Rep 30′ | Rep 90′ | Rep 180′ |

HR
(beats/min)

| | | | | | |
|---|---|---|---|---|---|
| Control | 275 ± 2 | 265 ± 9 | 266 ± 11 | 272 ± 12 | 266 ± 10 |
| Adenosine | 263 ± 7 | 276 ± 12 | 269 ± 14 | 261 ± 11 | 260 ± 11 |
| 150 | | | | | |
| Adenosine | 275 ± 8 | 279 ± 6 | 281 ± 9 | 262 ± 5 | 256 ± 4 |
| 300 | | | | | |
| Adenosine | 268 ± 2 | 274 ± 6 | 276 ± 5 | 266 ± 5 | 269 ± 5 |
| 400 | | | | | |
| MAP | | | | | |
| (mmHg) | | | | | |
| Control | 90.3 ± 3.7 | 78.3 ± 4.3 | 72.0 ± 6.5 | 78.0 ± 6.0 | 62.4 ± 4.4 |
| Adenosine | 88.3 ± 2.6 | 63.4 ± 5.3 | 60.1 ± 4.4 | 70.4 ± 5.6 | 74.0 ± 5.2 |
| 150 | | | | | |
| Adenosine | 99.5 ± 4.1 | 59.5 ± 5.4 | 57.5 ± 4.1 | 75.5 ± 2.3 | 77.8 ± 2.2 |
| 300 | | | | | |
| Adenosine | 94.5 ± 2.3 | 57.0 ± 2.8 | 52.8 ± 1.6 | 76.4 ± 2.1 | 75.8 ± 2.7 |
| 400 | | | | | |

TABLE 3

| | | | Heart weight | Risk zone | Infarct size |
|---|---|---|---|---|---|
| | n | Body weight (kg) | (g) | (cm³) | (cm³) |
| Control | 6 | 2.2 ± 0.1 | 6.8 ± 0.3 | 1.04 ± 0.08 | 0.38 ± 0.06 |
| AMP (30 min) | 5 | 2.0 ± 0.1 | 6.7 ± 0.1 | 0.89 ± 0.13 | 0.27 ± 0.05 |
| AMP (40 min) | 6 | 2.0 ± 0.0 | 6.7 ± 0.2 | 1.04 ± 0.09 | 0.27 ± 0.07 |
| AMP (70 min, late) | 6 | 2.0 ± 0.0 | 6.8 ± 0.2 | 0.97 ± 0.04 | 0.28 ± 0.07 |
| AMP (70 min, onset) | 6 | 2.1 ± 0.0 | 6.6 ± 0.1 | 1.04 ± 0.09 | 0.14 ± 0.03* |

TABLE 4

| | | Body | Heart | Risk zone | Infarct size | % infarction |
|---|---|---|---|---|---|---|
| | n | weight (kg) | weight (g) | (cm³) | (cm³) | of the risk zone |
| Control | 6 | 2.2 ± 0.1 | 6.8 ± 0.3 | 1.04 ± 0.08 | 0.38 ± 0.06 | 36 ± 3.1% |
| Adenosine 150 | 6 | 2.0 ± 0.1 | 6.6 ± 0.2 | 1.04 ± 0.11 | 0.36 ± 0.09 | 32.6 ± 5.8% |
| Adenosine 300 | 5 | 1.9 ± 0.0 | 6.7 ± 0.2 | 1.03 ± 0.06 | 0.36 ± 0.11 | 34.4 ± 9.5% |
| Adenosine 400 | 7 | 2.0 ± 0.1 | 6.7 ± 0.2 | 1.06 ± 0.09 | 0.30 ± 0.08 | 29.2 ± 8.0% |

TABLE 5

| | |
|---|---|
| | Infarct size |
| Infusion rate | (% of risk zone) |

| | | |
|---|---|---|
| 0 | | 36 ± 3.1% |
| 150 | ug/kg/min | 32.6. ± 5.8% |
| 300 | ug/kg/min | 34.4 ± 9.5% |
| 400 | ug/kg/min | 29.2 ± 8.0% |

A first embodiment according to the invention is a method of providing cardioprotection in a patient in need thereof comprising administering to said patient a pharmaceutically effective amount of a compound having adenosine A1/A2 agonistic activity, beginning at a time less than 10 minutes after the onset reperfusion, and continuing for a period of more than 30 minutes following the onset of reperfusion.

A second embodiment according to the invention is a method according to the first embodiment wherein the administering of the compound begins at the onset of reperfusion, and continues for a period of more than 30 minutes following the onset of reperfusion.

A third embodiment according to the invention is a method of providing cardioprotection in a patient in need thereof comprising administering to said patient a pharmaceutically effective amount of a compound having adenosine A1/A2 agonistic activity, beginning at a time 10 minutes or more before the onset of reperfusion, and continuing for a period of more than 30 minutes after the onset of reperfusion.

A fourth embodiment according to the invention is a method according to the first, second or third embodiment wherein the administering of the compound is continued for a period of more than 70 minutes after the onset of reperfusion.

A fifth embodiment according to the invention is a method of protecting against reperfusion injury in a patient in need thereof comprising administering to said patient a pharmaceutically effective amount of a compound having adenosine A1/A2 agonistic activity, beginning at a time less than 10 minutes after the onset reperfusion, and continuing for a period of more than 30 minutes following the onset of reperfusion.

A sixth embodiment according to the invention is a method according to the fifth embodiment wherein the administering of the compound begins at the onset of reperfusion, and continues for a period of more than 30 minutes following the onset of reperfusion

A seventh embodiment according to the invention is a method of protecting against reperfusion injury in a patient in need thereof comprising administering to said patient a pharmaceutically effective amount of a compound having adenosine A1/A2 agonistic activity, beginning at a time 10 minutes or more before the onset of reperfusion, and continuing for a period of more than 30 minutes after the onset of reperfusion.

An eighth embodiment according to the invention is a method according to the fifth, sixth or seventh embodiment wherein the adminstering of the compound is continued for a period of more than 70 minutes after the onset of reperfusion.

A ninth embodiment according to the invention is a method of protecting against ischemic injury in a patient in need thereof comprising administering to said patient a pharmaceutically effective amount of a compound having adenosine A1/A2 agonistic activity, beginning at a time less than 10 minutes after the onset reperfusion, and continuing for a period of more than 30 minutes following the onset of reperfusion.

A tenth embodiment according to the invention is a method according to the ninth embodiment wherein the administering of the compound begins at the onset of reperfusion, and continues for a period of more than 30 minutes following the onset of reperfusion

An eleventh embodiment according to the invention is a method of protecting against ischemic injury in a patient in need thereof comprising administering to said patient a pharmaceutically effective amount of a compound having adenosine A1/A2 agonistic activity, beginning at a time 10 minutes or more before the onset of reperfusion, and continuing for a period of more than 30 minutes after the onset of reperfusion.

A twelfth embodiment according to the invention is a method according to the ninth, tenth or eleventh embodiment wherein the administering of the compound is continued for a period of more than 70 minutes after the onset of reperfusion.

A thirteenth embodiment according to the invention is a method of providing cardioprotection prior to, during, or following cardiac surgery in a patient in need thereof comprising administering to said patient a pharmaceutically effective amount of a compound having adenosine A1/A2 agonistic activity, beginning at a time less than 10 minutes after the onset reperfusion, and continuing for a period of more than 30 minutes following the onset of reperfusion.

A fourteenth embodiment according to the invention is a method according to the thirteenth embodiment wherein the administering of the compound begins at the onset of reperfusion, and continues for a period of more than 30 minutes following the onset of reperfusion.

A fifteenth embodiment according to the invention is a method of providing cardioprotection prior to, during, or following cardiac surgery in a patient in need thereof comprising administering to said patient a pharmaceutically effective amount of a compound having adenosine A1/A2 agonistic activity, beginning at a time 10 minutes or more before the onset of reperfusion, and continuing for a period of more than 30 minutes after the onset of reperfusion.

A sixteenth embodiment according to the invention is a method according to the thirteenth, fourteenth or fifteenth embodiment wherein the administering of the compound is continued for a period of more than 70 minutes after the onset of reperfusion.

A seventeenth embodiment according to the invention is a method of providing cardioprotection prior to, during, or following ischemic attack in a patient in need thereof comprising administering to said patient a pharmaceutically effective amount of a compound having adenosine A1/A2 agonistic activity, beginning at a time less than 10 minutes after the onset reperfusion, and continuing for a period of more than 30 minutes following the onset of reperfusion.

An eighteenth embodiment according to the invention is a method according to the seventeenth embodiment wherein the administering of the compound begins at the onset of reperfusion, and continues for a period of more than 30 minutes following the onset of reperfusion

A nineteenth embodiment according to the invention is a method of providing cardioprotection prior to, during, or following ischemic attack in a patient in need thereof comprising administering to said patient a pharmaceutically effective amount of a compound having adenosine A1/A2 agonistic activity, beginning at a time 10 minutes or more before the onset of reperfusion, and continuing for a period of more than 30 minutes after the onset of reperfusion.

A twentieth embodiment according to the invention is a method according to the seventeenth, eighteenth or nineteenth embodiment wherein the administering of the compound is continued for a period of more than 70 minutes after the onset of reperfusion.

A twenty-first embodiment according to the invention is a method according to any one of the first through twentieth embodiment wherein the compound is of the formula

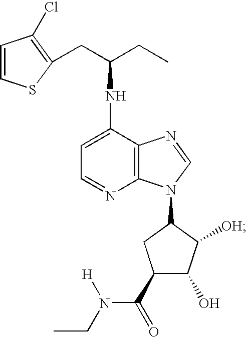

or a pharmaceutically acceptable salt thereof.

A twenty-second embodiment according to the invention is a method according to any one of the first through twentieth embodiments, wherein the compound is administered as a pharmaceutically acceptable salt thereof.

A twenty-third embodiment according to the invention is a method according to any one of the first through twenty-second embodiments, wherein the compound administered is contained in a pharmaceutical composition comprising a pharmaceutically acceptable carrier and a pharmaceutically effective amount of the compound.

In practice, the adenosine A1/A2 agonistic compounds administered in the methods according to the present invention may be administered parenterally, topically, rectally, transdermally, intrapulmonary or orally, but they are preferably administered parenterally and/or orally, and more preferably, parenterally.

Some compounds used in the method according to the invention may be basic, and such compounds are useful in the form of the free base or in the form of a pharmaceutically acceptable acid addition salt thereof.

Acid addition salts are a more convenient form for use; and in practice, use of the salt form inherently amounts to use of the free base form. The acids which can be used to prepare the acid addition salts include preferably those which produce, when combined with the free base, pharmaceutically acceptable salts, that is, salts whose anions are non-toxic to the patient in pharmaceutical doses of the salts, so that the beneficial inhibitory effects inherent in the free base are not vitiated by side effects ascribable to the anions. Pharmaceutically acceptable salts include those derived from mineral acids and organic acids, and include hydrohalides, e.g. hydrochlorides and hydrobromides, sulphates, phosphates, nitrates, sulphamates, acetates, citrates, lactates, tartrates, malonates, oxalates, salicylates, propionates, succinates, fumarates, maleates, methylene-bis-b-hydroxynaphthoates, gentisates, isethionates, di-p-toluoyltartrates, methane-sulphonates, ethanesulphonates, benzenesulphonates, p-toluenesulphonates, cyclohexylsulphamates and quinates.

Where the compound administered according to the methods of the invention is substituted with an acidic moiety, base addition salts may be formed and are simply a more convenient form for use; and in practice, use of the salt form inherently amounts to use of the free acid form. The bases which can be used to prepare the base addition salts include preferably those which produce, when combined with the free acid, pharmaceutically acceptable salts, that is, salts whose cations are non-toxic to the patient in pharmaceutical doses of the salts, so that the beneficial inhibitory effects inherent in the free base are not vitiated by side effects ascribable to the cations. Pharmaceutically acceptable salts, including those derived from alkali and alkaline earth metal salts, within the scope of the invention include those derived from the following bases: sodium hydride, sodium hydroxide, potassium hydroxide, calcium hydroxide, aluminium hydroxide, lithium hydroxide, magnesium hydroxide, zinc hydroxide, ammonia, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris(hydroxymethyl)aminomethane, tetramethylammonium hydroxide, and the like.

Suitable compositions containing compounds used according to the invention may be prepared by conventional means. For example, the compounds used according to the invention may be dissolved or suspended in a suitable carrier.

The compounds used according to the invention should be presented in forms permitting administration by the most suitable route, and the invention also relates to methods for providing cardioprotection by administering pharmaceutical compositions containing the compounds used according to the invention which are suitable for use in human or veterinary medicine. These compositions may be prepared according to the customary methods, using one or more pharmaceutically acceptable carrier, which comprise adjuvants or excipients. The adjuvants comprise, inter alia, diluents, sterile aqueous media and the various non-toxic organic solvents. The compositions may be presented in the form of tablets, pills, capsules, lozenges, troches, hard candies, granules, powders, aqueous solutions or suspensions, injectable solutions, elixirs or syrups, powders, solution or suspension for intrapulmonary administration and can contain one or more agents chosen from the group comprising sweeteners, flavorings, colorings, or stabilizers in order to obtain pharmaceutically acceptable preparations.

The choice of vehicle and the content of compounds used according to the invention in the vehicle are generally determined in accordance with the solubility and chemical properties of the compounds, the particular mode of administration and the provisions to be observed in pharmaceutical practice. For example, excipients such as sterile water, Ringer's solution, lactose, sodium citrate, isotonic saline solutions (monosodium or disodium phosphate, sodium, potassium, calcium or magnesium chloride, or mixtures of such salts), calcium carbonate and disintegrating agents such as starch, alginic acids and certain complex silicates combined with lubricants such as magnesium stearate, sodium lauryl sulfate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

For parenteral administration, emulsions, suspensions or solutions of the compounds used according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are useful. The solutions of the salts of the compounds used according to the invention are especially useful for administration by intramuscular, intravenous, intraarterial or subcutaneous injection or infusion techniques. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation or microfiltration.

The compounds having adenosine A1/A2 agonistic activity may also be formulated in a manner which resists rapid clearance from the vascular (arterial or venous) wall by convection and/or diffusion, thereby increasing the residence time of the composition at the desired site of action. Depot useful according to the invention may be in a copolymer matrix, such as ethylene-vinyl acetate, or a polyvinyl alcohol gel surrounded by a Silastic shell. Alternatively, the compound having adenosine A1/A2 agonistic activity may be delivered locally from a silicone polymer implanted in the adventitia.

An alternative approach for minimizing washout of the compounds during percutaneous, transvascular delivery comprises the use of nondiffusible, drug-eluting microparticles. The microparticles may be comprised of a variety of synthetic polymers, such as polylactide for example, or natural substances, including proteins or polysaccharides. Such microparticles enable strategic manipulation of variables including total dose of a drug and kinetics of its release. Microparticles can be injected efficiently into the arterial or venous wall through a porous balloon catheter or a balloon over stent, and are retained in the vascular wall and the periadventitial tissue for at least about two weeks. Formulations and methodologies for local, intravascular site-specific delivery of therapeutic agents are discussed, for example, in Reissen et al. (J. Am. Coll. Cardiol. 1994; 23: 1234-1244).

The medium for the compounds having adenosine A1/A2 agonistic activity can also be a hydrogel which is prepared from any biocompatible or non-cytotoxic (homo or hetero) polymer, such as a hydrophilic polyacrylic acid polymer that can act as a drug absorbing sponge. Such polymers have been described, for example, in application WO93/08845, the entire contents of which are hereby incorporated by reference. Certain of them, such as, in particular, those obtained from ethylene and/or propylene oxide are commercially available.

In addition, the compounds may be administered directly to the blood vessel wall by means of an angioplasty balloon which is coated with a hydrophilic film (for example a hydrogel), or by means of any other catheter containing an infusion chamber for the compounds, which can thus be applied in a precise manner to the site to be treated.

In the adult, the dosages of the adenosine A1/A2 agonistic compound are generally from about 0.00001 to about 0.5, preferably about 0.0001 to about 0.05, mg/kg body weight per day by inhalation, from about 0.0001 to about 1, preferably 0.001 to 0.5, mg/kg body weight per day by oral administration, and from about 0.00001 to about 0.1, preferably 0.0001 to 0.01, mg/kg body weight per day by intravenous administration.

The compound used according to-the invention may be administered as frequently as necessary in order to obtain the desired therapeutic effect. The dosage regimen in carrying out the method of this invention is that which insures maximum therapeutic response until improvement is obtained and thereafter the minimum effective level which gives relief. Some patients may respond rapidly to a higher or lower dose and may find much lower maintenance doses adequate. In selecting the appropriate dosages in any specific case, consideration must be given to the patient's weight, general health, age, and other factors which may influence response to the drug. Continuous parenteral infusion, in order to maintain therapeutically effective blood levels of the compound is also contemplated.

The present invention may be embodied in other specific forms without departing from the spirit or essential attributes thereof.

We claim:

1. A method of providing cardioprotection, or protecting against reperfusion injury, or protecting against ischemic injury, in a patient in need thereof comprising administering to said patient a pharmaceutically effective amount of a compound having adenosine A1/A2 agonistic activity, beginning at a time less than ten minutes after the onset reperfusion, and continuing for a period of more than 30 minutes following the onset of reperfusion.

2. A method according to claim 1, wherein the cardioprotection is provided prior to, during, or following cardiac surgery.

3. A method according to claim 1, wherein the cardioprotection is provided prior to, during, or following ischemic attack.

4. A method according to claim 1 wherein the administering of the compound begins at the onset of reperfusion, and continues for a period of more than 30 minutes following the onset of reperfusion.

5. A method according to claim 1, wherein the administering of the compound is continued for a period of more than 70 minutes after the onset of reperfusion.

6. A method according to claim 1, wherein the compound is of the formula 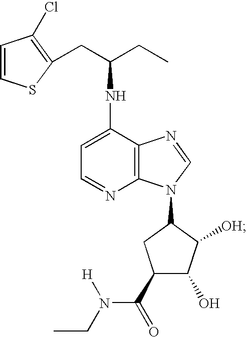
or a pharmaceutically acceptable salt thereof.

7. A method according to claim 1, wherein the compound is administered as a pharmaceutically acceptable salt thereof.

8. A method according to claim 1, wherein the compound administered is contained in a pharmaceutical composition comprising a pharmaceutically acceptable carrier and a pharmaceutically effective amount of the compound.

9. A method of providing cardioprotection, or protecting against reperfusion injury, or protecting against ischemic injury, in a patient in need thereof comprising administering to said patient a pharmaceutically effective amount of a compound having adenosine A1/A2 agonistic activity, beginning at a time 10 minutes or more before the onset of reperfusion, and continuing for a period of more than 30 minutes after the onset of reperfusion.

10. A method according to claim 9, wherein the cardioprotection is provided prior to, during, or following cardiac surgery.

11. A method according to claim 9, wherein the cardioprotection is provided prior to, during, or following ischemic attack.

12. A method according to claim 9, wherein the administering of the compound is continued for a period of more than 70 minutes after the onset of reperfusion.

13. A method according to claim 9, wherein the compound is of the formula 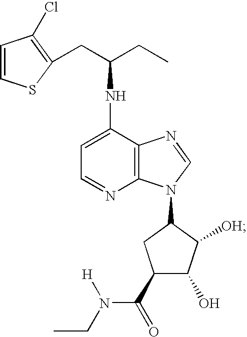
or a pharmaceutically acceptable salt thereof.

14. A method according to claim 9, wherein the compound is administered as a pharmaceutically acceptable salt thereof.

15. A method according to claim 9, wherein the compound administered is contained in a pharmaceutical composition comprising a pharmaceutically acceptable carrier and a pharmaceutically effective amount of the compound.
